(19) United States
(12) Patent Application Publication
(10) Pub. No.: US 2007/0102008 A1
(43) Pub. Date: May 10, 2007

(54) Table mounted thigh harness

(75) Inventor: Todd W. Sharratt, Stillwater, MN (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: The LeVahn Intellectual Property Holding Company, LLC, St Paul, MN (US)

(21) Appl. No.: 11/266,985

(22) Filed: Nov. 4, 2005

(51) Int. Cl. A61F 5/37

(52) U.S. Cl. 128/882

(57) ABSTRACT

An apparatus for securing a leg during a surgical procedure includes a support for mounting to a surgical table. A harness is positioned about a thigh and is secured to the support thereby supporting the thigh in an elevated position.

### BACKGROUND OF THE INVENTION

The present invention relates to a leg support for a surgical procedure. More particularly, the present invention relates to a table mounted harness for securing a knee in a flexed position for a surgical procedure.

Several methods have been developed for securing a knee in a flexed position for a surgical procedure such as a knee replacement surgery. The knee is preferably positioned into the flexed position to gain access to the ends of both the femur and tibia. Providing access to the ends of both the femur and tibia allows the damaged ends to be replaced with implants.

One method for securing the leg in a selected flexed position for a surgical procedure includes clamping the upper leg, also referred to as a thigh, to a surgical table with a clamping device that is positioned about the upper leg. With the thigh secured in the selected position, the knee joint is located proximate an edge of the surgical table. The lower leg hangs downward from the edge of the table, thereby flexing the knee for the surgical procedure.

Another method of securing the knee in a flex position for a surgical procedure is to insert a foot of the leg in a boot type structure attached to the table. The boot is moved while retaining the torso of the patient in a selected position, thereby causing the knee to flex into the selected position. Alternatively, the boot can be attached to the table and the position of the patient's torso is adjusted to cause the knee to flex into the selected position.

Another method of securing the knee in a flexed position is to provide a support apparatus that is positioned under the leg with the knee positioned in a selected flexed position. The leg is supported by the apparatus with the knee in the selected flexed position.

Utilizing any of the methods previously described to retain the knee in a selected flexed position requires a clinic or hospital to purchase a series of differently sized devices to accommodate patients of different sizes. Having to purchase a series of devices to perform the same procedure increases the costs to equip a clinic or hospital.

### SUMMARY OF THE INVENTION

The present invention includes an apparatus for supporting a leg during a surgical procedure. The apparatus includes a support for mounting to a surgical table and a harness for attaching to the support. The harness positions about the thigh and secures to the support thereby supporting the thigh in an elevated position.

### BRIEF DESCRIPTION OF THE DRAWINGS

FIG. 1 is a top view of a leg harness of the present invention attached to a thigh;

FIG. 2 is a perspective view of the leg harness of the present invention in a separated configuration;

FIG. 3 is another perspective view of the leg harness of the present invention in a separated configuration;

FIG. 4a is a partial perspective view of the leg harness of the present invention in a separated configuration;

FIG. 4b is a partial perspective view of the leg harness of the present invention in an attached configuration;

FIG. 5a is a partial sectional view of a leg support of the present invention in a separated configuration;

FIG. 5b is a partial sectional view of the leg support of the present invention in an attached configuration; and

FIG. 6 is a partial exploded view of the leg support of the present invention.

### DETAILED DESCRIPTION OF THE PREFERRED EMBODIMENTS

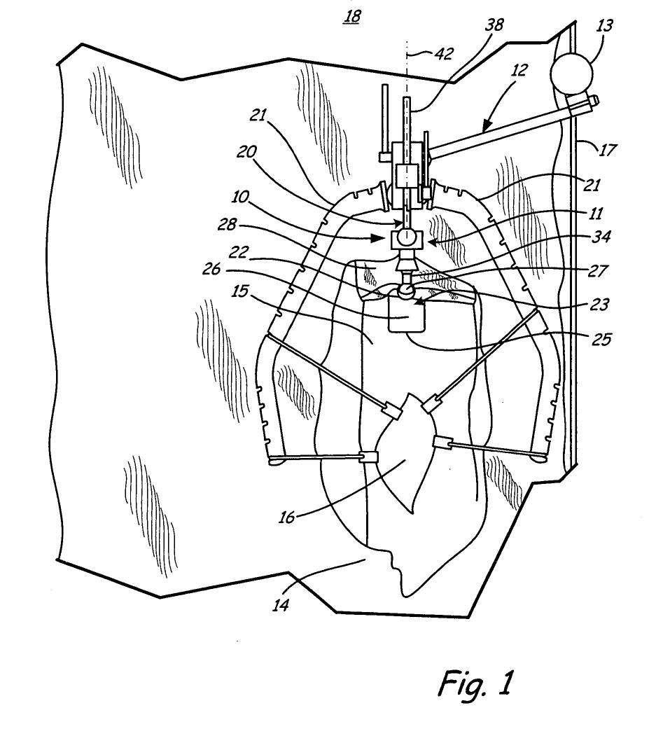

An apparatus for supporting a leg during a surgical procedure of the present invention is generally illustrated at 10 in FIG. 1. The apparatus 10 includes a leg support 11 that is mounted to a support apparatus 12 having arms 21 that are positioned on opposite sides of a leg 14 of a patient that is preferably bent at a knee joint 16. The support apparatus 12 is mounted to a post 13 that is attached to a rail 17 of a surgical table 18.

A harness 28 attaches the thigh 15 to the leg support 11. By harness is meant a fastener that retains an object, such as a thigh, in a selected position where at least a portion of the fastener has flexible characteristics. By flexible is meant capable of conforming to a surface of an object, such as a thigh.

The leg support 11 includes a shaft 20 that extends over the surgical table 18. A docking mechanism 23, such as a plate 26 attaches to a distal end 22 of the shaft 20. The plate 26 is positioned proximate the thigh 15 and preferably has an arcuate surface 25 that generally conforms to the shape of the thigh 15.

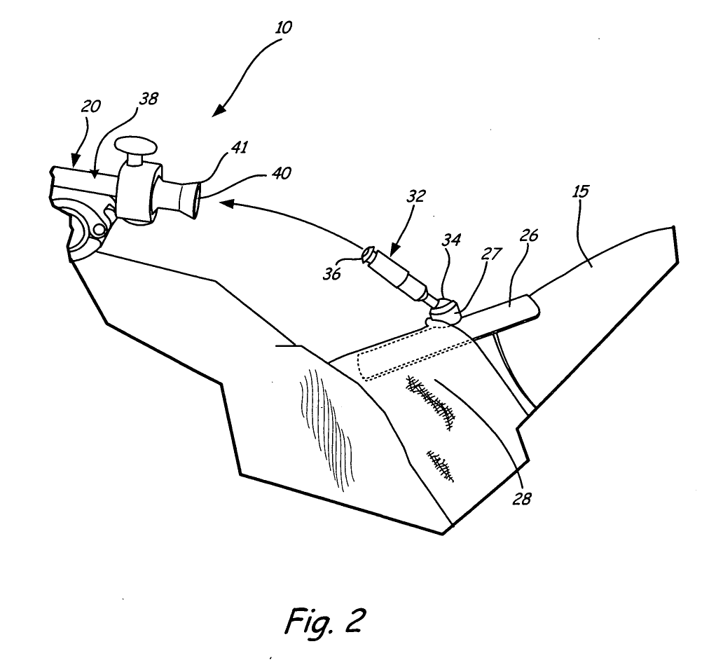

Referring to FIG. 2, the plate 26 is secured to the thigh 15 with the harness 28 that is positioned about a circumference of the thigh 15 and over at least a portion of the plate 26. The harness 28 secures the plate 26 on a selected position of the thigh 15 to provide access to a surgical site, such as the knee joint 16. Although the harness preferably attaches to the plate, it is with the scope of the present invention for the harness to attach to another docking mechanism 23 including, but not limited to, one or more bars, hooks, or loops.

An exemplary harness 28 includes surgical tape. However, other harnesses are also within the scope of the present invention including, but not limited to, a rope, a strap, a cloth strip, a mesh strip and an elastic bandage such as an Ace® bandage manufactured by Becton, Dickinson and Company of Franklin Lakes, N.J., and a polymeric material. The harness 28 may have elastic, extendible properties, rigid portions attached to flexible portions or non-elastic, flexible properties.

The harness 28 secures the thigh 15 to the plate 26 with any suitable attaching device such as a pressure sensitive adhesive, a loop and hook mechanism such as that sold under the VELCRO trademark, a buckle, a snap, a pin, a hook or a drawstring. Other fastening devices are also within the scope of the present invention.

The shaft 20 includes a stub shaft 32 that attaches to a main portion 38. The stub shaft 32 includes a pivot ball 34 that is positioned within a housing 27 that is attached to the plate 26. The housing 27 rotates about the pivot ball 34 to rotatably adjust a position of the plate 26.

The stub shaft 32, having a substantially circular cross-section, includes a proximal end 36 that positions within a substantially circular cross-section cavity 40. The cavity 40 opens from a distal end 41 of the main portion 38 substantially along a central axis 42. The distal end 41 of the main portion 38 outwardly flares to aid in positioning the stub shaft 32 within the cavity 40. A non-flared distal end 41 and a non-circular cross-section cavity 40 and stub shaft 32 are also within the scope of the present invention provided that the stub shaft 32 is positionable within the cavity 40.

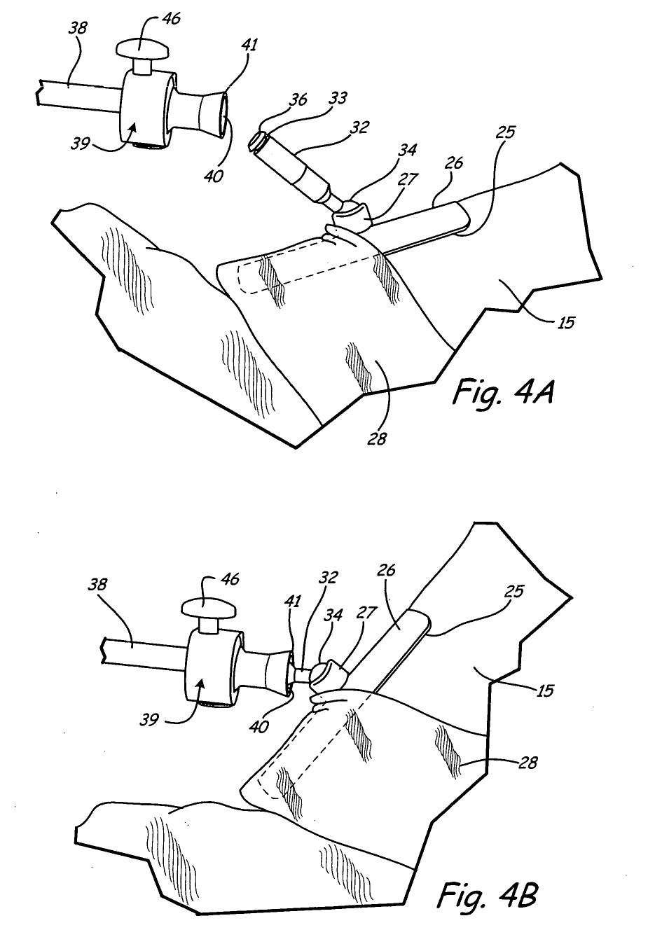

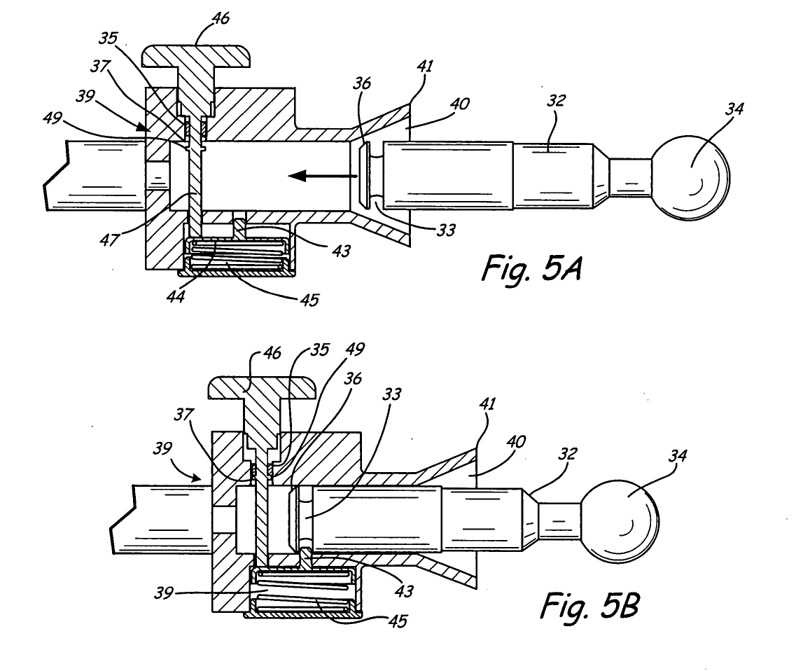

Referring to FIGS. 4A, 4B, 5A and 5B, a peg 43 extending from a plate 44 positions within an annular groove 33 in the stub shaft 32 to return the stub shaft 32 within the cavity 40, as best illustrated in FIGS. 4B and 5B. A compression spring 45 biases the plate 44 toward the cavity 40 to retain the peg 43 within the annular groove 33.

As best illustrated in FIGS. 4A and 5A, the peg 43 is displaced from the annular groove 33 by applying manual force to a knob 46 that is attached to a plunger 47 that engages the plate 44. As the plunger 47 engages the plate 44, manual force overcomes the bias of the compression spring 45 and causes the peg 43 to move from the cavity 40 and exit the annular groove 33.

The plunger 47 is positioned within a through bore 37 in the retaining mechanism 39 that intersects the cavity 40. The plunger 47 is retained within the through bore 37 through engagement of a shoulder 49 on the plunger 47 with a threaded nut 35 that threadably engages the threaded through bore 37.

With the peg 43 displaced from the annular groove 33, the stub shaft 32 disengages from the cavity 40 with manual force. The stub shaft 32 repositions within the cavity 40 by applying manual force to the knob 46 which forces the plunger 47 into the plate 44 and displaces the peg 43 from the cavity 40 by overcoming the bias of the compression spring 48. The stub shaft 32 repositions within the cavity 40 such that the annular groove 33 aligns with the peg 43. With the annular groove 33 aligned with the peg 43, manual force is removed from the knob 46 and the compression spring 48 forces the peg 43 into the annular groove 33 and thereby retains the stub shaft 32 to the main portion 38.

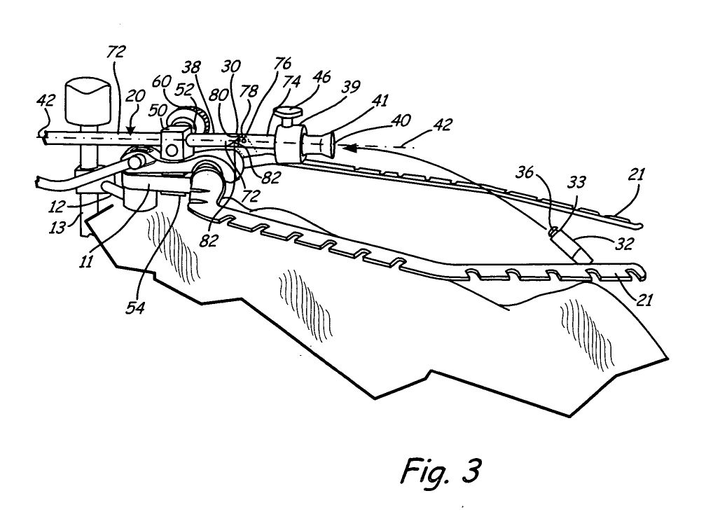

Referring to FIG. 3, the main portion 38 of the shaft 20 is slidably adjustable within a through bore 52 of a clamp 50 attachable to a support apparatus clamp 11. The support apparatus clamp 11 includes apertures that accept a post 54 extending from the clamp 50. The post 54 retains the clamp 50 to the support apparatus clamp 11. The post 54 and the surface defining the apertures preferably have flat surfaces that engage and prevent the post 54 from rotating within the apertures. However, it is within the scope of the present invention for the post 54 to rotate with respect to the support apparatus clamp 11.

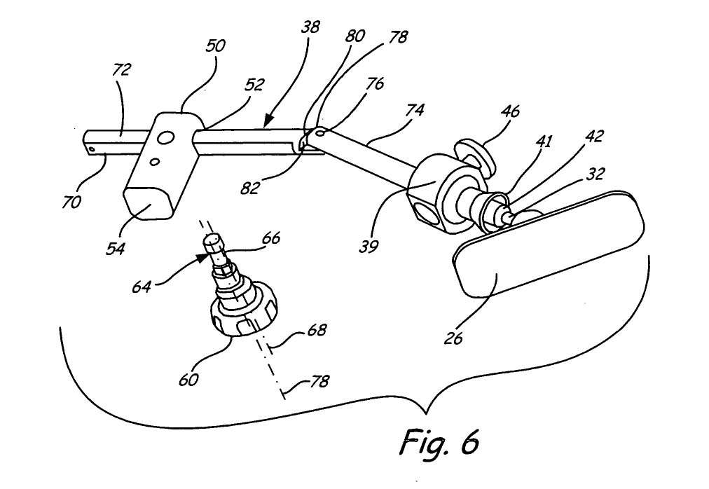

Referring to FIGS. 3 and 6, the main portion 38 is secured in a selected position by rotating a knob 60 attached to a camming mechanism 64. The mechanism 64 includes an eccentric portion 66 having an axis 68 offset from the axis 78 of rotation of the camming mechanism 64. As the camming mechanism 64 rotates, a surface of the eccentric portion 66 engages a substantially flat surface 70 on the main portion 38. The engagement of the surface of the eccentric portion 66 with the substantially flat surface 70 frictionally secures the main portion 38 in the selected position.

The main portion 38 preferably includes a proximal portion 72 having the flat surface 70 and a distal portion 74 pivotally attached to the proximal portion 72. However, the main portion 38 may also be constructed of a single member. A pivot pin 76 is positioned within aligned apertures (not shown) in the proximal portion 72 and the distal portion 74 to pivotally attach the distal portion 74 to the proximal portion 72. The pivot pin 76 frictionally engages the proximal portion 72 and the distal portion 74 such that manual force is required to adjust a position of the distal portion 74 with respect to the proximal portion 72.

A back surface 78 of the distal portion 74 engages a substantially vertical surface 80 on the proximal portion 72 to limit an upward movement of the distal portion 74. The back surface 78 of the distal portion 74 engages a slanted surface 82 of the proximal portion 72 to limit downward movement of the distal portion 74.

An angle defined by the substantially vertical surface 80 and the slanted surface 82 limits the rotational range of movement of the distal portion 74. The distal portion 74 may be adjusted within the range of movement to provide access to the cavity 40 when the thigh 15 is in the selected position.

In operation, the stub shaft 32 separates from the main portion 38 of the shaft 20 and the plate 26 is positioned in a selected position on the thigh 15. The plate 26 is secured to the thigh 15 with the harness 28, preferably with surgical tape. The harness 28 is wrapped around the circumference of the thigh 15 and also covers at least a portion of the plate 26 to secure the plate 26 to the thigh 15.

The post 54, extending from the clamp 50, is positioned within the apertures in the support apparatus clamp 11 to secure the clamp 50 in the selected position. Alternatively, the clamp 50 may be positioned in a selected position along the lengths of the support arms 21 and secured thereto with a clamp or another securing device.

With the clamp 50 secured in the selected position, the main portion 38 is slidably positioned within the through bore 52 in the clamp 50 to position the distal end 41 in the selected position. The camming mechanism 64 rotates to frictionally secure the main portion 38 in the selected position by engaging the flat surface 70 with the eccentric portion 66.

The thigh 15, having the stub shaft 32 secured thereto, is elevated by articulating the knee joint 16 and thereby positioning the proximal end 36 of the stub shaft 32 with the cavity 40 in the distal end 41 of the main portion 38. Manual force applied to the knob 46 overcomes the bias of the compression spring 48 to displace the peg 43 from the cavity 40. The proximal end 36 of the stub shaft 32 is positioned into the cavity 40 such that the annular groove 33 is aligned with the peg 43. Manual force is removed from the knob 46 such that the compression spring 48 biases the peg 43 into the annular groove 33 and thereby secures the stub shaft 32 to the main portion 38. With the stub shaft 32 secured to the main portion 38, the thigh 15 is secured in the selected elevated position with the knee joint 16 in a bent position.

To adjust the position of the thigh 15 and the knee 16, the camming mechanism 64 is rotated to release the frictional engagement between the surface of the eccentric portion 66 and the substantially flat surface 70 such that the main portion 38 is slidably positioned through the clamping bore 52. With the thigh 15 and the knee 16 in the selected position, the camming mechanism 64 is rotated and the main portion 38 is secured in the selected position with the frictional engagement. Additionally, the position of the thigh 15 is adjusted by pivoting the distal portion 74 of the main portion 38 about the pivot pin 76. By pivotally moving the distal portion 78, the thigh 15 is raised or lowered into a selected position.

Although the present invention has been described with reference to preferred embodiments, workers skilled in the art will recognize that changes may be made in form and detail without departing from the spirit and scope of the invention.

What is claimed is:

1. An apparatus for supporting a leg during a surgical procedure comprising:
a support for mounting to a surgical table;
a harness for attaching to the support and for securing a thigh thereto, the harness supporting the thigh in an elevated position

2. The apparatus of claim 1 and wherein the support comprises:
a shaft attached to the support apparatus and having a distal end proximate a thigh; and
a docking mechanism attached to the distal end of the shaft and the harness being engaged to the docking mechanism to secure the thigh in the elevated position.

3. The apparatus of claim 2 and wherein the docking mechanism rotatably attaches to the shaft.

4. The apparatus of claim 2 and wherein the shaft comprises:
a main portion attached to the support apparatus; and
a stub shaft attached to the main portion and the docking mechanism being attached to the stub shaft and wherein the stub shaft.

5. The apparatus of claim 4 and further comprising a retaining mechanism operably attaching the stub shaft to the main portion.

6. The apparatus of claim 2 and wherein the support further comprises a support apparatus, and wherein the support apparatus attaches the shaft to the surgical table.

7. The apparatus of claim 6 and further comprising a clamping mechanism attaching the shaft to the support apparatus.

8. The apparatus of claim 1 and wherein the harness comprises an adhesive tape.

9. A method of securing a leg in a selected position for a surgical procedure, the method comprising:
positioning a patient on a surgical table;
attaching a support to the surgical table;
elevating a thigh of the patient to a selected elevated position;
positioning a harness about the thigh; and
securing the harness to the support to retain the thigh in the selected elevated position.

10. A method of claim 9 and wherein securing the harness to the support comprises:
positioning a docking mechanism attached to a distal end of the support proximate the thigh; and
securing the harness to the docking mechanism.

11. A method of claim 9 and wherein attaching the support to the surgical table comprises:
attaching a post to the table wherein the post extends upwardly therefrom; and
attaching a leg support to the post wherein the leg support extends over the surgical table.

12. The method of claim 11 wherein attaching the leg support to the post comprises:
attaching a main portion of the leg support to the post; and
operably attaching a stub shaft to the main portion, and wherein the stub shaft comprises a docking mechanism for securing the harness thereto.

13. The method of claim 12 and wherein operably attaching the stub shaft to the main potion comprises:
manipulating a retaining mechanism within the main portion to accept and retain the stub shaft thereto.

14. An apparatus for securing a leg of a patient during a surgical procedure, the apparatus comprising:
a surgical table;
a support for mounting to the surgical table; and
a harness for attaching to the support and for securing a thigh thereto, the harness supporting the thigh in an elevated position.

15. The apparatus of claim 14 and wherein the support comprises:
a post attached to a rail attached to the surgical table and extending upwardly therefrom;
a retractor support apparatus attached to the post and extending over the table and about the leg of the patient; and
a shaft attached to the retractor support apparatus and having a docking mechanism attached to a distal end thereof and wherein the harness attaches to the docking mechanism for retaining the thigh in the elevated position.

16. The apparatus of claim 15 and further comprising a pivot ball attached to the distal end of the shaft and wherein the docking mechanism rotatably mounts to the pivot ball.

17. The apparatus of claim 15 and wherein the shaft comprises:
a main portion attached to the retractor support apparatus; and
a stub shaft comprising a distal end secured to the docking mechanism and a proximal end securable to the main portion.

18. The apparatus of claim 17 and further comprising a securing mechanism operably attached to the main portion and wherein the securing mechanism secures the stub shaft to the main portion.

19. The apparatus of claim 15 and further comprising a camming mechanism attached to the retractor support apparatus and wherein the camming mechanism rotates to secure the shaft in a selected position such that the thigh is secured in the selected elevated position for the surgical procedure.

20. The apparatus of claim 14 and wherein the harness comprises an adhesive tape.
